(19) United States
(12) Patent Application Publication
(10) Pub. No.: US 2026/0041085 A1
(43) Pub. Date: Feb. 12, 2026

(54) POACEAE POLLEN PRESERVATION USING DILUENTS BLENDED WITH STARCH

(71) Applicant: PowerPollen, Inc., Ames, IA (US)

(72) Inventors: Marc Albertsen, Johnston, IA (US); Alex Austin, Ames, IA (US); Federico Valverde, Polk City, IA (US); Mark Westgate, Ames, IA (US)

(73) Assignee: PowerPollen, Inc., Ames, IA (US)

(21) Appl. No.: 19/290,668

(22) Filed: Aug. 5, 2025

Related U.S. Application Data
(60) Provisional application No. 63/679,795, filed on Aug. 6, 2024.
(60) Provisional application No. 63/721,752, filed on Nov. 18, 2024.

(51) Int. Cl. A01N 3/00

(52) U.S. Cl. CPC A01N 3/00

(57) ABSTRACT

Disclosed is a method of preserving pollen and storing pollen in a diluent that contains between 10% and 50% starch granules by weight or by volume. The preservation technique maintains pollen viability such that the pollen can be used to pollinate plants and create seeds. The method results in stored pollen with improved viability as compared to pollen that is left in ambient conditions or that is mixed with other diluents that do not contain the starch component. The starch can be derived from any source. Also encompassed by the invention are compositions of pollen in diluent mixtures wherein the diluent contains between 10% and 50% starch granules by weight or by volume. The compositions include the separation of dead pollen contents from live pollen grains by using starch mixed with other solid particulates.

This application claims priority from U.S. Provisional Patent Application No. 63/679,795 filed Aug. 6, 2024 and titled Poaceae Pollen Preservation Using Diluents Blended with Starch, and from U.S. Provisional Patent Application No. 63/721,752 filed Nov. 18, 2024, also titled Poaceae Pollen Preservation Using Diluents Blended with Starch. The entire contents of U.S. Provisional Patent Application Nos. 63/679,795 and 63/721,752 are hereby incorporated by reference.

### FIELD OF THE INVENTION

This invention relates generally to a novel method for storing and preserving pollen that increases the overall viability and fertility of the pollen and prevents pollen necrosis, which results in improved pollen for use in pollinating plants. The invention may be conducted with either fresh pollen, partially dried pollen, or pollen that has previously been stored or preserved.

### BACKGROUND

The current invention has application to the field of pollen longevity and viability. Pollen longevity is significantly influenced by environmental conditions, most notably temperature and relative humidity. Pollen, which is naturally shed from the flowers or flowering structures of angiosperms, is subject to rapid loss of viability once it is shed from the plant. Viability can be lost in minutes to hours depending on species and environmental conditions. Exposure to dry air and high temperature is particularly detrimental to pollen viability and longevity once it is shed from the plant. Thus, under natural field conditions, pollen has a limited lifespan during which it remains viable, referred to in this application as the “viability window”, as provided herein below. In particular, pollen from the Poaceae (Gramineae) family of plants, commonly referred to as grasses, is particularly vulnerable and short-lived (Barnabas & Kovacs (1997) In: Pollen Biotechnology For Crop Production And Improvement. (1997). Sawhney, V. K., and K. R. Shivanna (eds). Cambridge University Press. pp. 293-314). This family of plants includes many economically important cereal crops, including maize. Methods to improve pollen viability and extend the duration of its viability are of significant value to the agricultural industry.

Specifically, if pollen collected from plants can be stored in a viable state for a period of time, this pollen may be used to pollinate female flowers as desired in a number of advantageous ways. Using stored pollen allows for pollination which is not dependent on active pollen shed, temporal synchrony with pistil (female flower) receptivity, use of male sterility, and/or physical isolation from other pollen sources. Currently, many species rely on self-pollination or cross pollination by neighboring plants to produce fertile seed or grain. Typically, in the agricultural hybrid seed industry, mechanical, physical, and/or genetic interventions are required to ensure female plants are cross pollinated, and not self-pollinated, so that pollen of a specific genetic constitution is employed to produce hybrid seed. Such measures, for example, are used routinely to produce hybrid maize and rice seed. In some crops, however, even these measures do not ensure cross pollination by a specific desired pollen source. Currently, it is not economical to produce these crops commercially as hybrids, although research continues in this regard. Examples of these crops include, but are not limited to, wheat and soybean.

Many attempts have been made to preserve pollen and extend its viability for pollinations beyond the time the pollen would remain viable if left exposed to uncontrolled ambient conditions. Among the grasses, studies with maize are exemplary of the progress made in pollen preservation. Many types of treatments have been tested for maintaining or extending maize pollen viability and/or fertility. Among them, the favorability of treating and/or storing maize pollen at high humidity and/or cold temperature has been reported by many.

Among the earliest accounts of maize pollen preservation (Andronescu, Demetrius I., The physiology of the pollen of Zea mays with special regard to vitality. Thesis for degree of Ph.D. University of Illinois. 1915), it was reported that pollen died in two to four hours in the absence of controlled environmental storage conditions.

Various methods have been used since then to attempt to improve maize pollen preservation efficacy. These include raising the relative humidity of the storage environment and storage at low temperatures (Pfahler, P. L. and Linskens, H. F., (1973) Planta, 111(3), pp.253-259; Frova, C. B. and Feder, W. A., (1979) Ann Bot, 43(1), pp.75-79). When high humidity (90% RH) and low temperature (4° C.) during storage are combined for pollen treatment, the capacity of maize pollen to germinate on artificial media is partially maintained for eight days (Sartoris, G. B., (1942) Am J Bot, pp.395-400). In some cases, it may be desirable to treat pollen so that it is dehydrated to various degrees, such as by vacuum drying or exposing pollen to a relative humidity and temperature (i.e., vapor pressure deficit) that causes water to diffuse out of the pollen (Jackson, M. A. and Payne, A. R. (2007) Biocontrol Sci Techn, 17(7), pp.709-719), Greenspan, L., (1977) J Res Nat Bur Stand, 81(1), pp.89-96).

Desiccation is known to have a direct impact on pollen viability. Barnabas ((1985) Ann Bot 55:201-204) and Fonseca and Westgate ((2005) Field Crops Research 94:114-125) demonstrated that freshly harvested maize pollen could survive a reduction in original water content of approximately 50%, but few pollen grains demonstrated viability or a capacity for normal pollen tube formation with an additional water loss beyond that level. Other methods of storing pollen for varying periods of time are known in the art, including freeze-drying, vacuum-drying, and storage in organic non-polar solvents. Limitations in the scalability of these pollen preservation techniques combined with the complex, non-portable equipment requirements, however, render these techniques completely impractical for use because they are unable to accommodate the large volumes of pollen necessary for field-scale applications.

Field conditioning maize pollen at high humidity and low temperature commonly helps to revive pollen of low viability and/or extend its longevity, whereby at least limited seed formation occurs following pollination of female plants (See, for example, U.S. Pat. No. 10,575,517). Although high humidity and low temperature slow the temporal decay of viability during storage of Poaceae pollen, optimizing these environmental conditions for preservation only postpones the complete loss of viability and fertility. Methods in addition to regulating humidity and temperature are needed to further enhance the longevity of stored pollen so that it can be used in commercial practice of supplemental pollination for improved seed and grain production. Further, regulating humidity and temperature in large-scale applications is technologically challenging and expensive. Simpler approaches would make supplemental pollinations much more feasible.

U.S. Pat. No. 5,596,838 from Greaves, et al., discloses a method of storing pollen that involves a reduction in moisture level by exposing pollen to reduced atmospheric pressures prior to storage. This technique prepared small quantities of pollen, such as from a single maize plant, for subsequent storage under sub-zero conditions. The methodology and mechanical system requirements, however, lacked the capacity to produce stored pollen in quantities large enough to enable commercial seed production or grain production applications. These requirements effectively negated any opportunity to advance the technology beyond research level investigations. Another Greaves et al. patent, U.S. Pat. No. 6,141,904 provides that pollen may be placed in a carrier material to increase flowability, such as a flowable powder. In addition, U.S. Pat. No. 2,669,066 suggests mixing pollen with substances having a high protein content for treatment and application, such as powdered egg albumin, powdered casein, or powdered milk in instances of short-term storage.

The availability of preserved viable pollen overcomes many of the production challenges faced by the hybrid seed industry. With respect to hybrid seed production, the availability of stored pollen for delivery to female flowers can eliminate many standard, costly practices of seed production including, but not limited to: planting male plants in proximity to, but separate from, female plants to enable hybridization, increasing field area allocated to male parent plants due to their low pollen production, isolating female parent plants from undesired pollen sources, and use of genetic, chemical, or mechanical male sterility of the female plants. These standard, costly practices divert field space and resources away from female parent plants which produce the seed or grain. Eliminating any one of these practices would have an immediate positive impact on seed yield per acre. Moreover, stored pollen can be applied at any time female parent plants are receptive to pollen. When pollen shed from male parent plants and pollen receptivity of female parent plants fail to coincide as planned (due to management, environment, or genetic variation), application of preserved, viable pollen ensures pollination of female parent plants at the optimal time. Pollination by undesired external (adventitious) sources of pollen or undesired self-pollination of female parent plants also can be reduced or eliminated by applying stored pollen of a desired type at the most appropriate time. Furthermore, production-level fields for parent seed increase are typically an acre or more in size, while hybrid production fields are typically 10 acres or more in size. Such fields require kilograms of pollen for efficient pollination, thereby demonstrating the need for methods that can enable the preservation and storage of large quantities of pollen.

Today, the genetic and commercial potential of a particular hybrid seed is determined at the beginning of a growing season by the genetics of the pollen-donating male parent plants and pollen-receiving female parent plants after they are planted together in the field. Using the embodiments of the present disclosure, however, a hybrid seed producer responding to changing market opportunities can decide at the time of pollination to use a different male parent pollen (i.e., genetic source) for pollination to produce more valuable hybrid seed. Thus, stored pollen can be used to deliver unique genetic traits or genes that enhance seed quality characteristics to highly productive female parent inbreds. For example, traits might be delivered for resistance to select insect pests that currently are prevalent in the area. Importantly, the embodiments of the present disclosure also ensure a high level of genetic purity in the hybrid seed. Furthermore, the pollen preserved using the embodiments of the invention can be used to supplement self- or sib-pollen in grain production, or to deliver traits in cross-pollinated grain production.

Mixing pollen with a diluent can extend the viability of pollen storage by limiting deleterious pollen-pollen interactions. Diluents also can improve the uniformity of pollen delivery to receptive female flowers thereby increasing seed set, regardless of whether pollen is applied by hand or by machine. In addition, using diluents enables pollen to be applied in less-than-saturating doses to increase pollination efficiency and to distinguish effects of experimental treatments. As such, methods to improve pollen viability and extend the duration of its viability using novel diluents are of significant value to the agricultural industry.

### BRIEF DESCRIPTIONS OF THE FIGURES

FIG. 1 is a box plot showing kernel set (y axis) resulting from pollinations with pollen-diluent mixtures varying in lactose/starch content (x axis).

FIG. 2 is a box plot showing germination percentage of maize pollen plotted on the y-axisresulting from pollinations with pollen-diluent mixtures varying in lactose/starch content (x axis).

FIG. 3 is a box plot showing germination percentage of maize pollen plotted on the y-axis resulting from pollinations with pollen-diluent mixtures varying in lactose/starch content with the starches sourced from a variety of plant species (x axis).

FIG. 4 is a box plot showing percent germination of maize pollen plotted on the y-axis resulting from pollinations with pollen-diluent mixtures varying in lactose content mixed with various percentages of wheat and rice starch (x axis).

FIG. 5 is a box plot showing kernel set (y axis) resulting from pollinations with pollen stored in diluents containing various proportions of cassava starch. 100% lactose (0% starch) and 20% corn starch in lactose treatments are provided for comparison.

FIG. 6 is a box plot showing kernel set (y axis) resulting from pollinations with pollen stored in diluents containing various proportions of potato starch. 100% lactose (0% starch) and 20% corn starch in lactose treatments are provided for comparison.

FIG. 7 is a box plot showing kernel set (y axis) resulting from pollinations using pollen stored in diluents containing various proportions of mung bean starch. 100% lactose (0% starch) and 20% corn starch in lactose treatments are provided for comparison.

FIG. 8 is a box plot showing kernel set (y axis) per ear of maize resulting from pollinations using pollen stored in diluents containing various proportions of pea starch. 100% lactose (0% starch) and 20% corn starch in lactose treatments are provided for comparison.

### SUMMARY OF THE INVENTION

Provided is an invention comprising a method of preserving Poaceae pollen comprising mixing collected Poaceae pollen with a diluent comprising between 10% and 50% starch granules by weight or by volume, wherein said preserved Poaceae pollen maintains its viability and can be used to successfully pollinate plants. The method includes storing the Poaceae pollen mixed with the blended diluent. The storage can take place at a temperature ranging from −196 degrees Celsius to −80 degrees Celsius, at a temperature ranging from −80 degrees Celsius to 0 degrees Celsius, or at a temperature ranging from 0 degrees Celsius to 25 degrees Celsius. The diluent may comprise 15% to 25% starch granules by weight or by volume.

The invention encompasses the use of freshly collected Poaceae pollen or previously collected and preserved Poaceae pollen. The Poaceae pollen may have been obtained from a single genetic source or from multiple genetic sources, which may be combined prior to preservation.

Embodiments of the invention include applying the Poaceae pollen and diluent mixture to receptive plants. The application of the Poaceae pollen and diluent mixture can be used to generate seeds for seed production purposes or for grain production purposes.

The starch used in the diluent of the invention may be obtained from any source, including natural sources, chemical and transgenic sources, and synthetically derived starch. The starch used in the embodiments may be extracted from a root vegetable, such as potato, sweet potato, cassava, taro, and yam. The starch may be extracted from a grain, such as from corn, barley, buckwheat, wheat, and rice. The starch may be sago starch that has been extracted from a palm plant, or it may be arrowroot starch extracted from a plant such as Maranta arundinacea, Zamia integrifolia, Manihot esculenta, Tacca leontopetaloides, and Pueraria lobata. Furthermore, the starch may be extracted from a legume, such as from chickpea, cowpea, lentil, mung bean, pea, pigeon pea, fava bean, lima bean, and kidney bean.

The remaining components of the diluent may comprise solid particulates that prevent the interaction of live pollen grains with dead or dying pollen contents or grains, and may comprise organic or inorganic particles. For example, the diluent may comprise a monosaccharide, disaccharide, or trisaccharide. In some embodiments, the diluent comprises starch and lactose.

The method of the invention results in preserved Poaceae pollen with enhanced performance and enhanced stability when compared to preserved Poaceae pollen that has been stored in a similar diluent that is lacking the starch.

The invention also encompasses compositions, such as a population of Poaceae pollen grains mixed with a diluent comprising between 10% and 50% starch granules by weight or by volume, or between 15% and 25% starch granules by weight or by volume. The composition may be stored for a minimum of 12 hours prior to use in pollinating plants.

### GLOSSARY

“Vessel” is used herein to mean an enclosure suitable for containing and storing pollen.

“Dead pollen contents” is used herein to mean any material related to a dead or dying pollen grain, including but not limited to the pollen grain itself and material leaked from a dead or dying pollen grain.

“Female parent plant” or “female plant” is used to mean a plant that is being used as the recipient of pollen, and which has receptive flowers that are being fertilized.

“Fertile” or “fertility” is used to describe the ability of pollen to deliver the sperm nuclei to the ovule and thereby effect double fertilization. In flowering plants, the term “double fertilization” refers to one sperm nucleus fusing with the polar nuclei to produce the endosperm tissues, and the other sperm nucleus fusing with the egg nucleus to produce the embryo.

“Fresh” when applied to pollen means pollen released from the anthers of a flower which, in its natural pattern of organ growth and development, releases pollen upon dehiscence in response to promotive environmental conditions.

“Germinability” refers to the ability of pollen to germinate and form a pollen tube.

“Longevity,” when applied to pollen, is used describe the length of time that pollen remains both viable and fertile.

“Loss of viability” is a pollen characteristic that means that the viability level of the pollen has fallen to a level below that required for successful initiation of seed development.

“Loss of fertility” is a pollen characteristic that means that the fertility level of the pollen has fallen to a level below that required for successful initiation of seed development.

“Male parent plant” or “male plant” means a plant from which pollen is collected from for use in pollinations.

“Preservation” means any storage of collected pollen that results in a level of viability, fertility, or both, which is different than the level of viability, fertility, or both, which would occur if the pollen were held in unregulated conditions.

“Root vegetable” means any underground plant part used as a temporary storage organ containing starch and other nutrients that also can be consumed by humans or animals. In agricultural terminology, the term applies to true roots such as taproots and tuberous roots as well as bulbs, corms, stolons, rhizomes, and tubers.

“Storage” means any period of pollen containment with the intent of using the pollen at a later time and/or date.

“Surface-to-surface contact” between pollen grains means the touching of any part of the surface area of one pollen grain to the surface area of one or more other pollen grains.

“Viable” or “viability” is used to describe pollen that is able to germinate and grow a pollen tube to at least a length twice the diameter of the pollen grain or pollen which has been judged viable by demonstration that the cellular nature of the material remains integral and is judged to maintain intactness such that normal cellular processes of metabolism and intracellular functioning is possible. The viability of pollen can be assessed in numerous ways, including, but not limited to, assessment of pollen tube growth on artificial media or excised stigmas or styles, assessment of cellular intactness by vital staining of numerous sorts, absence of electrolyte (e.g., potassium) leakage, and impedance flow cytometry. Viability can refer to a single pollen grain or a population of pollen grains. When a percentage value is used to describe pollen viability, the value is typically being applied to a population of pollen.

“Viability window” refers to the lifespan during which pollen remains viable when exposed to unregulated conditions.

“Vigor” refers to pollen performance, such as speed of germination and tube growth.

### DETAILED DESCRIPTION

The following is a detailed description of embodiments of technology and methods of storing and/or preserving pollen which enable improved and/or extended viability of collected pollen by subjecting it to specialized storage conditions and techniques. This invention disclosure describes a novel set of diluents which include starch. These blended diluents enhance pollen viability 4-fold or more in storage as assessed by kernel set and/or pollen germination. Experimentation with lactose blended with starch has demonstrated that the beneficial range of starch in lactose is 10 to 50% by weight. These results are particularly surprising because storing pollen in 100% starch as a diluent prevents seed set and pollen germination completely. The benefit of the starch and lactose blended diluent is concentration dependent, reaching a maximum benefit at 20 to 30% starch (w/w). Furthermore, the starch can be derived from a variety of sources. In addition, the starch-blended diluents of the invention are highly beneficial and readily applicable for the agricultural industry since they can be employed without any concerns for biodegradability, environmental degradation, equipment deterioration, or personal safety.

The pollen that is combined with the diluent mixture may be collected from actively shedding plants or, alternatively, the pollen may have been previously collected and stored according to other methods known in the art now or in the future. Such methods include, but are not limited to, partial drying, freezing, freeze-drying, storing in liquid nitrogen, or following the embodiments of U.S. Pat. No. 11,344,027, Cereal Crop Pollen Field Conditioning Method or U.S. Pat. No. 11,844,348, Pollen Preservation and Storage Method.

According to one embodiment of the invention, in order to preserve pollen with improved and/or extended viability, the pollen is mixed with a diluent comprising between 10% and 50% starch. The starch component may be combined with other suitable pollen diluents, including, but not limited to, sugars such as lactose. Chemical desiccating or sequestering agents may also be added to the diluent mixture. Experimentation has shown that lactose works particularly well when combined with starch to provide an effective diluent that extends the viability and consistency of performance of stored Poaceae pollen.

Dead pollen contents have been proven to cause live pollen grains to die more quickly than they would if the live pollen grains were separated from the dead pollen contents (see, for example, U.S. Pat. No. 12,245,587). Evidently, cellular components leached from the dead pollen interact with viable pollen grains and causes their death or loss of viability. The present invention seeks to minimize and/or prevent such interaction. U.S. Pat. No. 12,245,587 uses solid particulates to separate pollen grains and separate live pollen grains from interactions with dead pollen contents. The present invention demonstrates that when a specific percentage of starch is mixed with a solid particulate diluent and then used in pollen preservation, there are surprising and unexpected benefits.

Collected pollen stored in the diluent of the present invention may be stored for any length of time, including but not limited to just long enough to transfer pollen to a plant in the same or a neighboring field, minutes, hours, days, weeks, months, and/or years. For example, in some cases, the pollen is stored in the diluent of the present invention for 30 minutes, for 1 hour, for 2 hours, for 3 hours, for 4 hours, for 5 hours, for 8 hours, for 10 hours, for 12 hours, for 15 hours, for 20 hours, for 24 hours, for 2 days, for 3 days, for 5 days, for a week, for several weeks, for a month, for several months, for a year, or for several years. In the embodiments of the invention, surface to surface contact between grains of pollen and/or the cellular content of grains of pollen is prevented or limited.

For the purposes of this invention, the pollen and diluent mixture can be stored in a vessel of any shape, size, and construction suitable for the containment of the pollen and diluent mixture, such as a container, a cartridge, an enclosure, a tube, or any other type of vessel. Furthermore, the vessel may optionally be aerated and may also optionally be housed within a controlled environment. For example, the vessel may optionally be stored within a refrigerated environment, a freezer, a cooler, or any other environment in which the temperature may be controlled.

The starch used in the diluent mixture can be derived from any plant type or plant organ that accumulates starch as temporary carbohydrate storage. For example, it can be extracted from various plant types, including but not limited to root vegetables, grains, legumes, and palms. In particular, root vegetable starches are useful in the practice of the invention, including, for example, starch extracted from potato, sweet potato, cassava, taro, and yam. Likewise, grain starches may be used in the practice of the invention, such as, for example, starch extracted from corn, barley, buckwheat, wheat, and rice. Another starch that is useful for the practice of the invention is sago starch. Sago starch is extracted from the pith of various tropical ‘sago palms’ such as Metroxylon sagu and other Metroxylon species, but also from cycad species, in particular, Cycas revoluta. In addition, arrowroot starch can be used in the practice of the invention. Arrowroot starch is obtained from the rhizomes of various tropical plants, including, but not limited to, Maranta arundinacea, Zamia integrifolia, Manihot esculenta, Tacca leontopetaloides, and Pueraria lobata. Starch extracted from legume seeds may also be used in the practice of the invention. For example, mature seeds of chickpea, cowpea, lentil, mung bean, pea, and pigeon pea may be particularly useful in the practice of this invention as they are comprised of 40 to 60% starch (Tayade, et al (2019) Frontiers Plant Science Vol 10. <doi: 10.3389/fpls.2019.01213>).

Furthermore, starches used in embodiments of the invention may be unique genetic variant starches or transgenically developed or modified starches. Additionally, the starch may be entirely artificially synthesized rather than being extracted or derived from a plant. Accordingly, starches used in the practice of the invention may be naturally, chemically, or transgenically derived or created.

It is beneficial that the diluent mixtures of the invention regulate pollen moisture content. Furthermore, the diluents may provide increased flowability when the pollen is applied to a female plant, such as during mechanical application. It is also desirable to use diluents that are cost effective for large scale operations, which is one benefit of solid substances or particulates generally. Another benefit of solid particulates is that they are more easily portable or transportable, which can be especially beneficial when the method is performed on pollen as it is collected, such as in a field. It is a benefit of the present invention that the diluents are not harmful to the plant and/or the environment. Furthermore, it is beneficial that the diluent mixture does not interfere with the normal function of the stigmas of the female parent plant onto which the pollen will be applied. Similarly, it is beneficial that the diluent and the pollen grains can separate during or immediately after application, such that the diluent doesn't bind to the pollen and/or female parent plant, including but not limited to pollen receptors, during application to female parent plants.

It is a benefit of the present invention that the diluent mixture that includes starch can impart an improvement or enhancement in pollen performance, stability, and viability compared to the same diluent that is lacking the starch component. When used without the starch present, some diluents maintain or enhance pollen performance or stability, but the use of starch as a blended component of the diluent shows a surprising and unexpected benefit. As further demonstrated in the working examples of this disclosure, this benefit is observed whether pollen viability is assessed in terms of pollen germination in vitro or in terms of kernel set measured in planta.

The concentration of starch in the diluent mixture may vary depending on a number of factors including, but not limited to, the type of pollen being preserved, the type of starch(es) being used in the diluent mixture, and the nature of the other solid particulates in the diluent mixture. The starch may be added as a percentage of the mixture by weight or by volume. One of skill in the art will understand and appreciate that the decision to measure the starch component by weight or by volume will depend on the nature and density of the other components of the diluent mixture. In the case of the blends used in the working examples of this disclosure, the starch was typically blended by weight.

As discussed above, the methods disclosed herein improve and/or extend the viability of pollen in storage. Viable pollen can successfully germinate and commonly possesses the vigor necessary to promote fertilization and initiation of seed development. Not all viable pollen is also fertile pollen. In some cases, even when a pollen grain germinates and commences with pollen tube growth, it may lack the vigor necessary to reach the ovule and promote fertilization. Non-viable pollen grains cannot successfully germinate. Thus pollen with good viability is desirable for use with the methods in the present disclosure. In maize, the level of viability and fertility required for successful pollinations is typically accepted or defined in the art to be an average of four grains of fresh pollen per maize silk, or four to ten grains of preserved pollen which has been preserved according to the methods of the present disclosure.

As discussed above, many embodiments of the present invention require collection of freshly shed pollen or acquiring pollen which has previously been collected. Pollen collection for the purposes of the invention may take place by any method and in any location where plants are grown, such as a growth chamber, a greenhouse, a glasshouse, a shade house, a hoop house, a vertical farming facility, a hydroponic facility, or any growing facility providing cultured tassels, as described below. Pollen may, optionally, be field-conditioned prior to storage or preservation, such as according to the methods set forth in U.S. Pat. No. 10,575,517. Field conditioning, if used, may also take place using other methods known in the art, now or in the future. In some embodiments, field conditioning may occur immediately upon collection or at any point between collection and storage. In addition, it is anticipated that field conditioning may include methods of the present invention.

The collection of fresh pollen may be conducted in ways commonly known in the art. For example, pollen may be collected from freshly shedding flowers or male flower structures produced in any variety of manners. In the case of maize, and many other species, the plant is monoecious and contains separate male and female inflorescences on a single plant. In the practice of breeding, pollination, cross-pollination, and hybridization, some plants act as the male parent plant from which pollen is collected for use in pollinations, and some plants act as the female parent plant being the recipient of the pollen. In the case of self-pollinations, a single plant is acting as both the male parent and the female parent because the female flowers are fertilized by pollen from its own male flowers. Similar to self-pollinations, sib-pollinations occur when plants of the same genetics (such as a neighboring plant from the same field) act as both the male parent and the female parent. For purposes of this disclosure, the term “self-pollination” includes sib-pollinations, as the same genetics are involved in both situations. Still referring to maize as an example, pollen is collected from freshly shedding male flowers borne on tassels, which may be attached or detached from the plant.

Pollen from any type of plant may be collected from plants grown in any environment suitable for plant growth. Such environments include, but are not limited to, a field, a growth chamber, a greenhouse, a glasshouse, a shade house, a hoop house, a vertical farming facility or a hydroponic facility. Alternatively, pollen may be collected directly from anthers by crushing or grinding the anthers or by letting them naturally continue to expel pollen as the anther dries, thereby releasing the pollen and allowing for its collection. In addition, pollen may be collected from a tassel culture facility (Pareddy et al. (1989) Theor Appl Genet 77:521-526). Mature tassels removed from plants grown in any type of controlled environment facility or the field are maintained under controlled conditions that promote pollen shed. Also, pollen can be collected from cultured tassels derived from tissue harvested from immature flower structures and developed into tassels that produce viable pollen.

Alternatively, anthers may be collected directly, instead of pollen. Anthers may be collected prior to the pollen maturing in the anther, during the maturation time, or when the pollen is mature and being released. Whole anthers may also be stored for later use by storing them in the compositions of the invention. Pollen that is either immature, partially mature, close to mature but not yet being released, or mature dehisced pollen, may be released from collected anthers by stripping, grinding, shaking, drying, or other similar methods. Grinding is particularly effective for immature pollen because the grinding of the entire anther along with the immature pollen can be conducted in the composition of the invention. This process will automatically release the pollen into the ideal environment where the surfaces of each pollen grain are limited in their ability to touch other grains. Moreover, anthers may be stored employing methods of the present invention, including but not limited to, with solid, liquid, gas, and/or physical separation of pollen grains to separate dead pollen contents from live pollen grains.

This disclosure outlines both the methods of minimizing surface-to-surface contact between pollen grains, and the diluents that are used to minimize pollen surface-to-surface contact to improve viability and fertility. The diluent may be refreshed or replaced over time, including with the same or a new diluent. Accordingly, this may include switching pollen from one diluent to another during storage.

Embodiments of the invention utilize solid particles to separate pollen grains from dead pollen contents and/or each other, thus preventing interaction between dead pollen contents and live pollen grains. In many embodiments, the solid particles minimize surface-to-surface contact between grains of pollen. In the case of solid particle substances, preferably the size of the particles should be determined in relation to the size of the pollen grains intended to be stored. If the solid particles are too large, they can sometimes be ineffective at preventing contact between pollen grains because the pollen can collect in pockets between the particles. If the solid particles are too small, they can also be ineffective at separating the pollen grains and dead pollen contents because they do not provide enough separation. Different species of plant pollen can vary in size, ranging from approximately 5 to more than 125 μm in diameter. In addition, some pollen grains have surfaces that are not perfectly round which result in a larger surface area. Accordingly, using solid particle compositions that contain a range of particle sizes and/or shapes will allow use with pollen from different species. Alternatively, routine experimentation will easily determine the ideal size of solid particles based upon the size and morphology of the pollen grains to be stored.

A further consideration is the ratio of solid particulate diluent to pollen grains. High ratios of diluent to pollen tend to increase separation between dead pollen contents and live pollen grains. Moreover, higher ratios result in more dilution of the pollen, which is sometimes beneficial for application of the pollen to the female plants. In some examples of embodiments, the ratio may be anywhere from 1:1 to 1:100 of pollen grains to diluent. In some examples, a ratio of 1:4 pollen grains to diluent is used. Moreover, the ideal ratio can depend on the amount of time for which the pollen will be stored. Over the short term, the ratio has been shown to be less important. On the other hand, as time progresses, the ratio becomes more important in maintaining viability of preserved pollen. The ratio can be optimized for benefits including, but not limited to, enhancing storage duration, diluting pollen, and/or optimizing flowability.

Preferably, the nature of any solid particle composition used to separate dead pollen contents from live pollen grains includes particles that do not cause negative interactions with the pollen. In some embodiments, the solid particles are of a non-reactive nature so that the pollen is essentially unaffected by the particles. For example, hydrophilic or hygroscopic solid particles must not remove too much moisture from the pollen; the loss of moisture impacts the viability of pollen, with greater moisture loss resulting in greater viability loss, and eventually the death of the pollen grain. A solid particle with a slight hydrophilic or hygroscopic nature, however, can be beneficial because it can separate dead pollen contents from live pollen grains. More specifically, and without being bound to a particular theory, experimentation has shown that substances that are slightly hydrophilic absorb dead pollen contents that escape or leak from dead and/or dying pollen. Because the hydrophilic substances absorb this material, it is separated from live pollen grains. Moreover, slightly hydrophilic materials help regulate pollen moisture content, which can oftentimes be important to pollen viability. Slightly hydrophilic substances often do not desiccate pollen in the same way that more hydrophilic substances may. Hydrophobic particles may be less desirable because they may have a natural tendency to avoid physical contact with the pollen grains which could result in a gradient between the pollen and the particle. Nonetheless, some hydrophobic substances can be beneficial. Namely, some hydrophobic substances may attach to the surface of pollen grains and at least partially coat the grains. Such coating can result in separation of dead pollen contents from live pollen grains and/or separation between living pollen grains.

Starch is hygroscopic. It can absorb moisture up to 10-17% of its weight when equilibrated at normal atmospheric conditions (Leach, 1965, Starch: Chemistry and Technology. Vol. 1. New York: Academic Press; p. 20.). It is commonly used as an absorbent in drug formulations to keep powders dry and ensure the stability of drugs that are liable to deteriorate by hydrolysis and other chemical reactions. Starch is one of the most widely used pharmaceutical excipients because it is nontoxic, odorless, inexpensive, widely available, and biocompatible. It is widely used as a binder in the wet granulation process to ensure uniform dosages in tablets. Starch also is a convenient and effective disintegrant of tablets due to its natural swelling properties in the presence of water (Olobayo, 2020, Chemical Properties of Starch. IntechOpen. doi: 10.5772/intechopen.89811).

When a starch grain is exposed to water, the water molecules pass through the surface of the starch granules into the interior. As water molecules continue to penetrate, they eventually reach the amorphous region of the starch granules and hydrate the amylose and amylopectin chains. This hydration causes starch granules to expand (Zhang et al., 2024, Trends in Food Science & Technology 144: 104321). The capacity of native starch to absorb water depends on the physico-chemical properties of the starch grain and its components. These properties include the molecular composition of the starch, granule surface structure, long-range crystalline structures, short-range ordered structures, and hydrophilic groups. In particular, the amylopectin (branched) chain length and amylose content are positively correlated with starch water absorption capacity. Likewise, the presence of large numbers of concavities, cracks, and holes on the starch surface accelerates water penetration into the starch granules, resulting in a higher water absorption rate.

While not wishing to be bound to any one theory, it is postulated that the benefits of including starch in a diluent in the proper proportion may serve to stabilize the moisture content of the storage environment that contains recalcitrant pollen grains at 50% to 60% moisture. Too little starch allows the pollen to retain moisture and continue to respire rapidly; too much starch serves to accelerate pollen drying and rapid loss of viability.

Accordingly, pollen moisture content and regulation of same is beneficial in certain embodiments of the invention. In some examples of embodiments, the method may include pollen moisture content at 15-60%. More specifically, pollen moisture content may be 40-58%. In even further embodiments, pollen moisture content may be 45-55%. In some embodiments of the invention, it has been found that the pollen can be kept at higher moisture levels than has previously been considered optimal in the art. Accordingly, in some embodiments, it can be beneficial if substance used in the invention can enable maintenance of higher pollen moisture content without detriment.

Solid particles that can be mixed with starch to form diluents useful for the disclosed invention include inorganic or organic particles, including, but not limited to the following: dormancy regulators, osmotic regulators, cellular respiration inhibitors and disruptors, electron transport inhibitors, uncouplers of membrane electron transport, membrane stabilizers and conditioners, cell wall stabilizers and conditioners, growth regulators, plant hormones, cryoprotectants, monosaccharides, disaccharides, polysaccharides, anti-flocculants, dispersal agents, bentonite, cellulose, sand, desiccants, flow agents, dry sugars, and activated charcoal, or any combinations or mixtures thereof. It is to be understood that the selection of the solid particles to be combined with the starch may be influenced by the type of pollen to be stored. For example, the selection may be based on size and/or how tolerant specific pollen is to certain interactions and characteristics. An ideal composition may include a single solid particle type or a combination of particle types that are chosen to maximize pollen viability and longevity. It may also contain a range of particle sizes to ensure that both larger and smaller grains of pollen are being separated with equal efficiency.

In addition, it may be beneficial to add a substance that will increase flowability of the stored pollen such that the pollen can be easily applied to plants at a later date. Such a compound must not cause negative interactions between other solid particles in the storage medium, nor between the pollen grains. Examples of compounds that separate dead pollen contents from live pollen grains and also aid in flowability include, but are not limited to, various forms of silica, clays, talc, zeolite, magnesium chloride, and stearic acid.

Pollen can be subjected to additional preservation steps to increase its ability to retain viability and fertility over storage periods. The preparation of pollen for storage requires a careful process including an environment with known relative humidity levels, as well as specific temperature and atmospheric pressure conditions. For example, depending upon the preparation methods used, the temperature may be −80° C. to about 25° C. In some embodiments, relative humidity may be 50-100%. Moreover, air pressure may be 15 kPa-150 kPa. In addition, the addition or removal of certain gases will assist in maximizing storage success. Such gases may include but are not limited to nitrogen, carbon dioxide, nitrous oxide, oxygen, or a mixture of these gases.

Optionally, during the period of pollen storage, the pollen and starch diluent mixture contained within the storage vessel may be agitated mechanically. Agitation may be occasional or continuous. In some embodiments, agitation is continuous. The agitation serves to expose the maximum surface area of the pollen to the diluent, thereby ensuring a homogenous mix of the pollen with the diluent and preventing surface-to-surface contact of pollen grains, clumping of the pollen, and the spread of pollen necrosis. The continued, regular, and/or occasional agitation of pollen grains in the composition, including dead or dying pollen grains, may be achieved in a number of ways, including the use of vibration, forced air, rotation, mixing, or other means.

The temperature of the storage vessel or the environment in which the storage vessel containing the pollen composition is stored may be an ambient temperature. Alternatively, the pollen composition may be refrigerated or frozen, or maintained in cryogenic storage. Pollen that is frozen or stored cryogenically may need to be appropriately prepared for such storage. The preparation may depend on the species of pollen to be stored. Many pollen species must be dried to a specific pollen moisture content before they can be successfully stored at temperatures below freezing. For example, the pollen composition of the invention may therefore be preserved and stabilized using techniques such as those disclosed in U.S. Patent Application Publication No. 2024/0237637. Alternatively, the pollen may be frozen or cryogenically stored and mixed with the diluent after being removed from storage and acclimated. Accordingly, the compositions of the invention may be maintained at temperatures ranging from about −196° C. to about 30° C. Temperatures at which the pollen in the diluent can be stored include −196° C., −150° C., −120° C., −80° C., −70° C., −60° C., −50° C., −40° C., −30° C., −20° C., −10° C., 0° C. 1° C. to about 10° C., including about 1° C., about 2° C., about 3° C., about 4° C., about 5° C., about 6° C., about 7° C., about 8° C., about 9° C. about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., and about 25° C. In some cases, the pollen in the diluent can be stored at higher temperatures, including temperatures ranging from about 10° C. to about 25° C. While lower temperatures are often preferable, this invention also has the ability to maintain and/or increase pollen viability at temperatures higher than those that have previously been disclosed in the art.

In examples of embodiments, pollen subjected to the disclosed method may be used in any application where pollen is a commercial or experimental unit. In one example, the preserved pollen may be used to produce seed (whether hybrid, parent, or otherwise) in any setting, including but not limited to a laboratory, greenhouse, and field. In another example, the pollen may be used to produce grain (whether hybrid or otherwise) in any setting, including but not limited to a laboratory, greenhouse, and field. The preservation techniques disclosed in this invention are intended to successfully preserve pollen such that the preserved pollen maintains its viability to the extent that a sufficient number of viable pollen grains are delivered to stigmas to ensure successful fertilization. For example, in corn, about 4 to about 10 grains of pollen per stigma (silk) are sufficient for successful fertilization.

Although various representative embodiments of this invention have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

The following examples illustrate the present invention in more detail and are illustrative of how the invention described herein could be implemented in pollen of various species.

Example 1: Seed set resulting from pollinations using maize pollen stored in diluent mixtures of lactose and corn starch.

This study was conducted in July 2023 on field-grown plants in Ankeny, IA to evaluate the longevity of maize pollen stored in various diluent mixtures of lactose and starch for three days at 5° C. in sealed containers. Fresh pollen collected from Alexho hybrid was mixed with the diluents in a 1:4 ratio of pollen: diluent by weight. Pollen-diluent mixtures were applied by hand (scoop and tube method) to female plants in amounts that limited kernel set. Pure pollen was applied to control plants at 4 times the rate to assess potential kernel set of similarly aged plants (3 reps of 8 plants each per treatment). The resulting kernel number per ear was evaluated at least 14 days after pollination. Diluents tested include: 100% lactose, 100% corn starch, 97% lactose blended with 3% corn starch, 95% lactose blended with 5% corn starch, 90% lactose blended with 10% corn starch, and 80% lactose blended with 20% corn starch by weight.

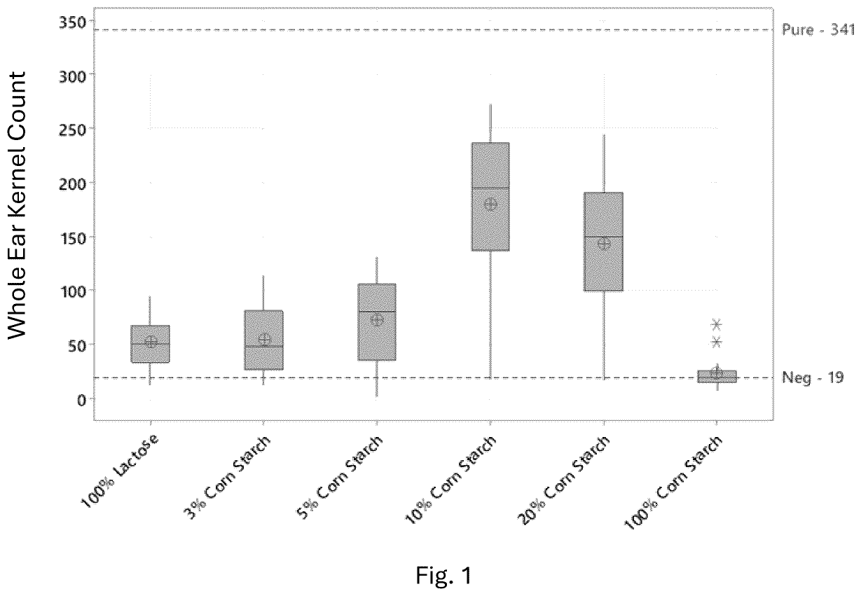

FIG. 1 shows that when fresh pollen is stored for three days in 100% lactose diluent, pollinations conducted using that stored pollen result in seed set significantly above the negative (unpollinated) controls, as expected from numerous previous studies. Substituting a proportion of this diluent with corn starch had a variable impact on seed set, depending on the concentration of starch in the diluent. Diluents containing 10 to 20% starch increased seed set 3 to 4-fold over the 100% lactose diluent. Lower concentrations of starch were far less effective, and 100% corn starch completely failed to maintain pollen viability. The dramatic improvement in viability (as measured by the ability to affect fertilization) when pollen was stored in a diluent containing 10 to 20% corn starch was a completely novel and unexpected discovery, given the limited benefit of the pure diluent components on maintaining pollen viability in this experiment.

Example 2: Comparison of diluent mixtures containing corn, potato, or cassava starch.

This study was conducted in February 2024 in Ames, IA to evaluate the longevity of maize pollen stored in various diluents containing starch obtained from various plant sources. Fresh pollen collected from inbred PHM81 growing in a controlled environment was mixed with lactose and starch blended diluents in a 1:4 pollen: diluent ratio by weight. The mixture was then stored for three days at 5° C. in sealed containers. Diluents evaluated included: 97% lactose blended with 3% corn starch, 100% potato starch, 80% lactose blended with 20% potato starch, 100% cassava starch, and 80% lactose blended with 20% cassava starch by weight. Maintenance of pollen viability in storage was determined by in vitro germination. Pollen grains were counted as germinated if an intact pollen tube emerged and was longer than the diameter of the pollen grain after one hour incubation at 20° C. in germination media.

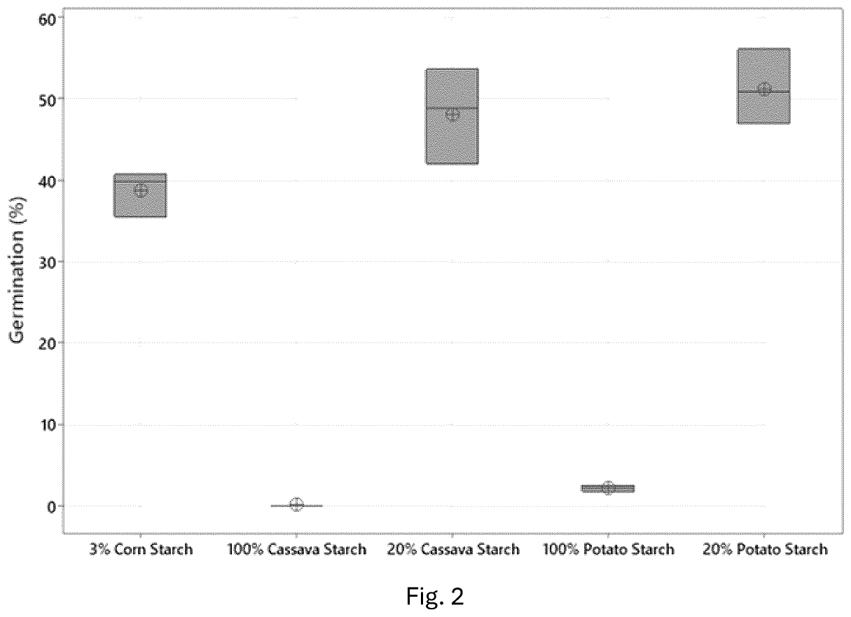

FIG. 2 shows the percent germination of maize pollen observed after storage for three days in lactose diluent containing starch from maize, cassava, or potato. As observed in Example 1, storing pollen in 97% lactose blended with 3% corn starch maintained pollen viability, as measured by emergence of a pollen tube in vitro. Storing pollen in potato starch or cassava starch at 20% of the lactose diluent mixture also maintained pollen viability, albeit to a greater extent than the diluent containing 3% corn starch. Similar to the results presented in Example 1, these data indicate the addition of plant starch to a lactose diluent at 20% by weight (w/w) had a dramatic positive impact on maintaining the viability of maize pollen in storage, regardless of the source of starch.

This result is highly unexpected because these seed-and tuber-sourced starches vary dramatically in starch granule size, granule crystalline and surface morphology, amylopectin-amylose content, and wetting characteristics (Remya, R, & Jyothi A N (2015) Journal of Root Crops 41:37-47, Tarique J, et al. (2022) Polymers (Base1) <doi: 10.3390/polym14030388>, Moorthy N (2002) Starch 54:559-592). These results also are quite surprising since cereal and tuber starches vary dramatically in phosphorous content, which directly impacts starch granule microstructure, hydration capacity, and their surface physico-chemical properties (Carciofi M, et al (2011) Journal of Cereal Science 54:339-346).

Example 3: Viability of maize pollen stored in diluent mixtures containing 20, 50% or 100% more starch.

This study was conducted in February 2024 in Ames, IA to evaluate whether concentrations of starch greater than 20% by weight in the diluent mixture could be beneficial for extending pollen viability in storage. As in Example 2, pollen viability was assessed by quantifying percent germination in liquid media. Fresh pollen collected from inbred PHM81 growing in a controlled environment was mixed with lactose and starch diluents in a 1:4 pollen:diluent ratio by weight and stored for three days at 5° C. in sealed containers. Starches were sourced from maize kernels, cassava tubers, potato tubers, and arrowroot stolons. Each source of starch was evaluated in lactose diluent mixtures at 0/100, 50/50 and 80/20 (lactose/starch) ratios by weight.

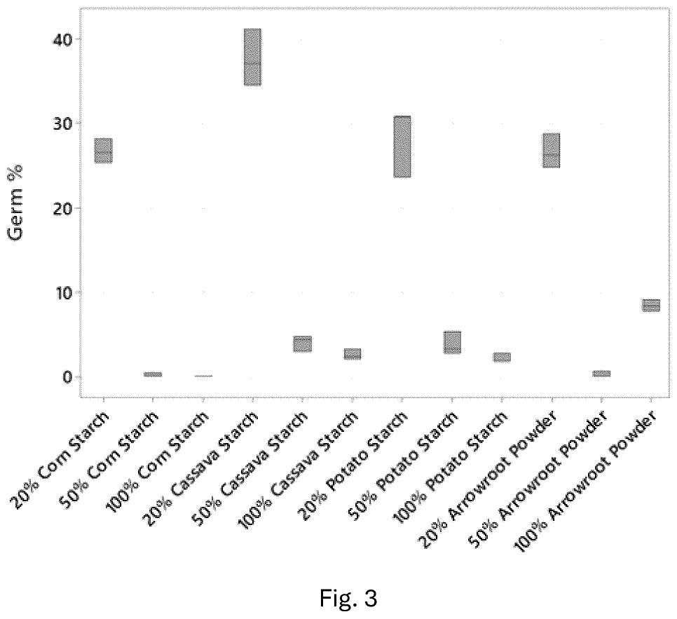

FIG. 3 shows that diluent mixtures containing 20% starch were superior for maintaining pollen viability in storage than any diluent mixtures containing 50% or more starch, regardless of the source of starch in the diluent. These results confirm that including 20% starch (w/w) in the diluent is highly beneficial for maintaining pollen viability in storage. That the physico/chemical benefit of starch in this proportion of diluent is consistent across a diverse range of plant-based starches is an entirely novel and unexpected result. Lactose-based diluents containing 50% or more starch are detrimental to viability of pollen in storage.

Example 4: Viability of maize pollen stored in diluent mixtures containing starch derived from maize, wheat, and rice kernels.

This study was conducted in Santa Isabel, Puerto Rico during March 2024 to evaluate the longevity of maize pollen stored in various diluent mixtures for three days at 5° C. in sealed storage vessels. Pollen viability was assessed as percent germination in vitro. Diluent mixtures containing lactose combined with starch derived from maize, wheat, or rice were evaluated, with starch comprising 100, 80, 50, and 20 percent of the diluent mixture by weight. Fresh pollen collected from ILLHO inbred was mixed with these diluents in a 1:4 ratio of pollen: diluent by weight. Pollen viability was assessed by in vitro germination. Pollen grains were considered germinated if an intact pollen tube emerged and was longer than the diameter of the pollen grain after one hour incubation at 20° C.

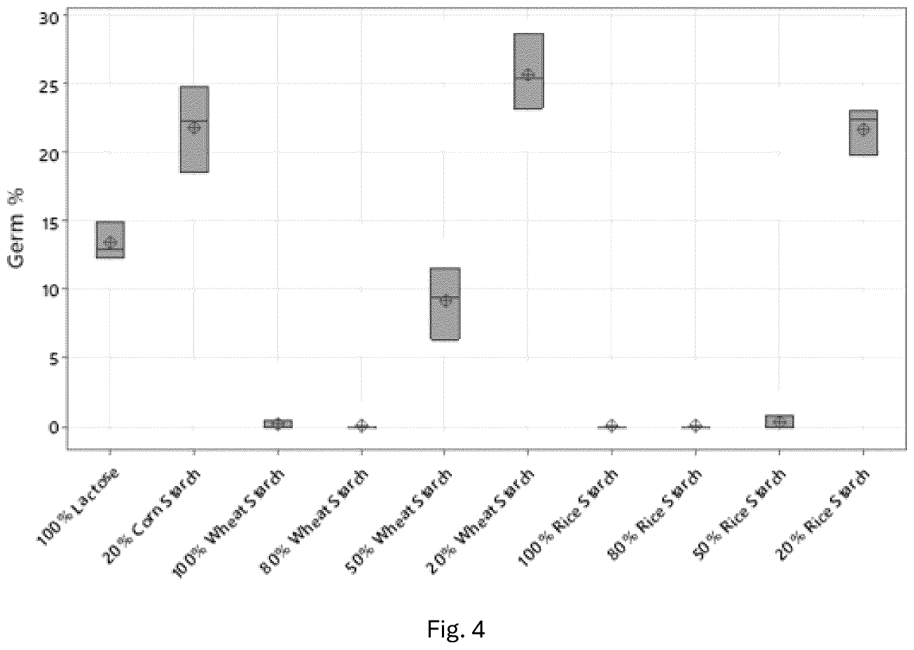

FIG. 4 shows that diluent mixtures containing 20% starch were far superior for maintained pollen viability in storage than any diluent mixtures containing 50% or more starch, whether the starch was derived from maize, wheat, or rice kernels. As observed in Examples 1 and 2, including 20% starch (w/w) in the diluent mixture is highly beneficial for maintaining pollen viability in storage. That the benefit of starch in this proportion of diluent is consistent across the three cereal species that produce the vast majority of the global starch supply is remarkable and entirely unexpected.

Example 5: Kernel set using pollen stored in diluent mixtures containing starch derived from maize kernels and cassava tubers.

This study was conducted in Santa Isabel, Puerto Rico during February 2024 to evaluate the longevity of maize pollen stored in diluent mixtures containing starch for three days at 5° C. in sealed storage vessels. Diluent mixtures containing lactose blended with starch derived from maize kernels or cassava tubers were evaluated. The starch component comprised 100, 80, 50, and 20 percent of the diluent mixture by weight. Fresh pollen collected from ILLHO inbred was mixed with diluents in a 1:4 ratio of pollen:diluent by weight. Pollen viability was assessed after 3 days of storage in terms of kernel set per ear on inbred R5357 resulting from metered hand pollinations (scoop and tube method) designed to expose treatment effects by limiting kernel set per ear. Pollen: diluent mixtures were applied to 10 female plants approximately three days after first silk emergence, with three repetitions (n=30). Kernels formed per ear were measured approximately 14 days after pollination.

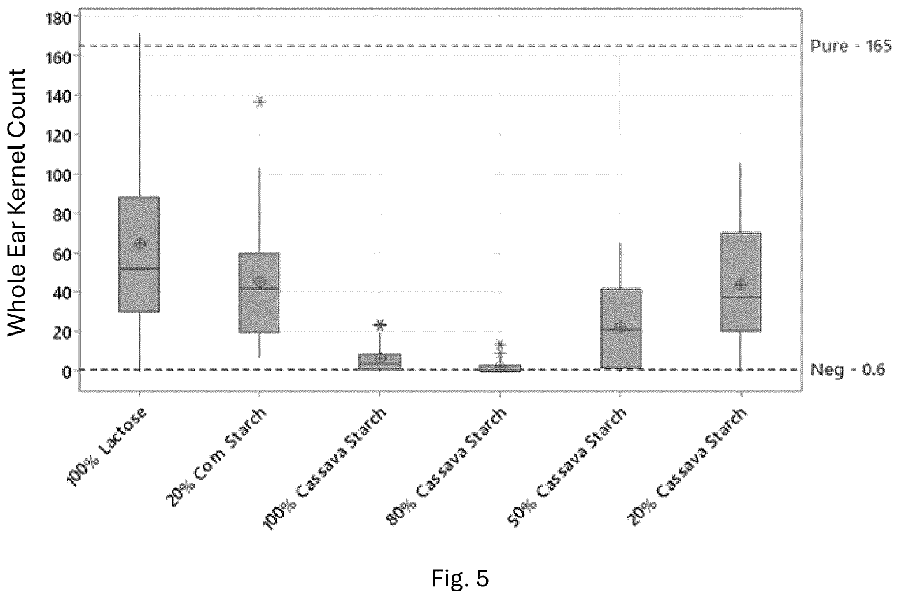

FIG. 5 shows that storing pollen in diluent mixture containing with 80% lactose blended with 20% corn starch, 80% lactose blended with 20% cassava starch, or 100% lactose all maintained pollen viability in storage better than did diluents containing starch in excess of 50% by weight. These results confirm the benefit of including starch as a modifying component of diluent mixtures, as indicated by in vitro germination results presented in Examples 2, 3, and 4. The positive and consistent impact on seed set could not be expected from the in vitro germination experiments (Examples 2, 3, and 4) as seed formation requires both pollen germination and pollen vigor to be maintained in storage and to be expressed under challenging field conditions.

Example 6: Kernel set using pollen stored in diluent mixtures containing starch derived from maize kernels and potato tubers.

This study was conducted in Santa Isabel, Puerto Rico during February 2024 to evaluate the longevity of maize pollen stored in diluent mixtures containing starch for three days at 5° C. in sealed storage vessels. Diluent mixtures containing lactose blended with starch derived from maize kernels or potato tubers were evaluated. The starch component comprised 100, 80, 50, and 20 percent of the diluent mixture by weight. Fresh pollen collected from ALEXHO inbred was mixed with diluents in a 1:4 ratio of pollen: diluent by weight. Pollen viability was assessed after 3 days of storage in terms of kernel set per ear on inbred R5357 resulting from metered hand pollinations (scoop and tube method) designed to expose treatment effects by limiting kernel set per ear. Pollen: diluent mixtures were applied to 10 female plants approximately three days after first silk emergence, with three repetitions (n=30). Kernels formed per ear were measured approximately 14 days after pollination.

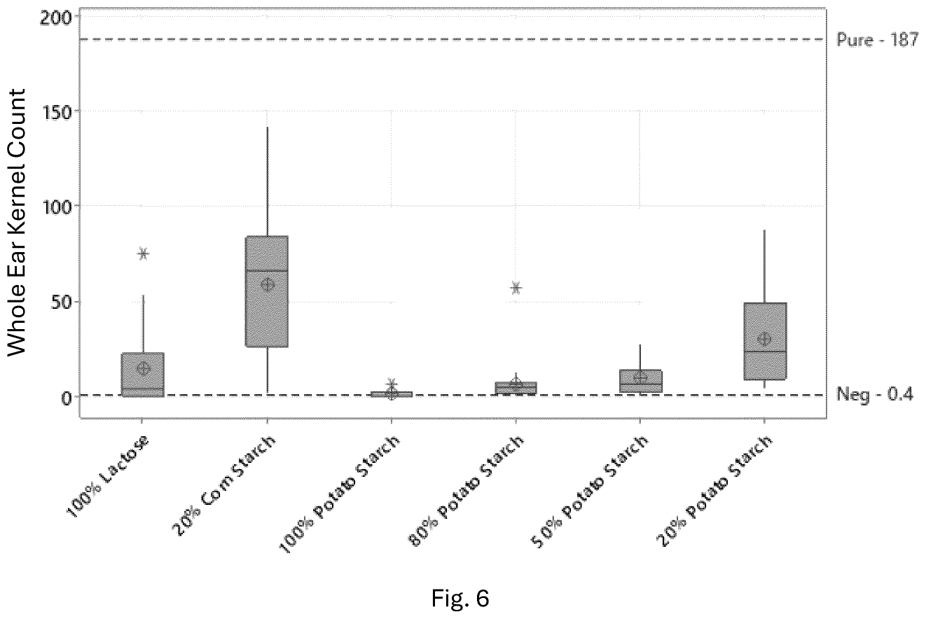

FIG. 6 shows that storing pollen in diluent mixtures containing 20% corn starch or 20% potato starch maintained pollen viability in storage better than did diluents containing potato starch in excess of 50% by weight. These results confirm the benefit of including starch as a modifying component of the diluent mixture to maintain kernel set, as observed in Example 5 using starch derived from maize and cassava. Similarly, the positive and consistent impact on seed set reflected an unexpected improvement in pollen germination and vigor expressed under challenging field conditions.

It is also noteworthy and quite unexpected that including starch (whether kernel-derived or tuber-derived) in the diluent mixture at a concentration greater than 10% but less than 50% (w/w) maintained pollen viability more consistently than did the pure lactose diluent alone, whether pollen viability was assessed in terms of pollen germination in vitro or kernel set measured in planta.

Example 7: Field evaluation of pollen-diluent mixtures including mung bean (Vigna radiata) starch.

This study was conducted on field-grown plants in Ankeny, Iowa during August 2024. The study objective was to evaluate the longevity of maize pollen stored in diluents comprising lactose blended with mung bean starch. Fresh pollen collected mechanically from maize inbred WT944-6 was mixed with the various starch-blended diluents in a 4:1 (diluent:pollen) ratio by weight. Starch blended diluents included the following blends (by weight): 80% lactose combined with 20% corn starch; 100% mung bean starch; 80% lactose combined with 20% mung bean starch; 50% lactose combined with 50% mung bean starch; and 20% lactose combined with 80% mung bean starch. Diluent: pollen mixtures were stored at 4° C. in 1 L storage vessels. After three days of storage, pollen-diluent mixtures were applied in metered (kernel limiting) doses onto exposed silks of male-sterile inbred F0457 approximately three days after silks first emerged. Pollinations were performed by hand (scoop and tube technique using a 1/64^{th} teaspoon of pollen-diluent mixture) to 10 female plants per treatment/rep with three replications (n=30 per treatment, n=180 total).

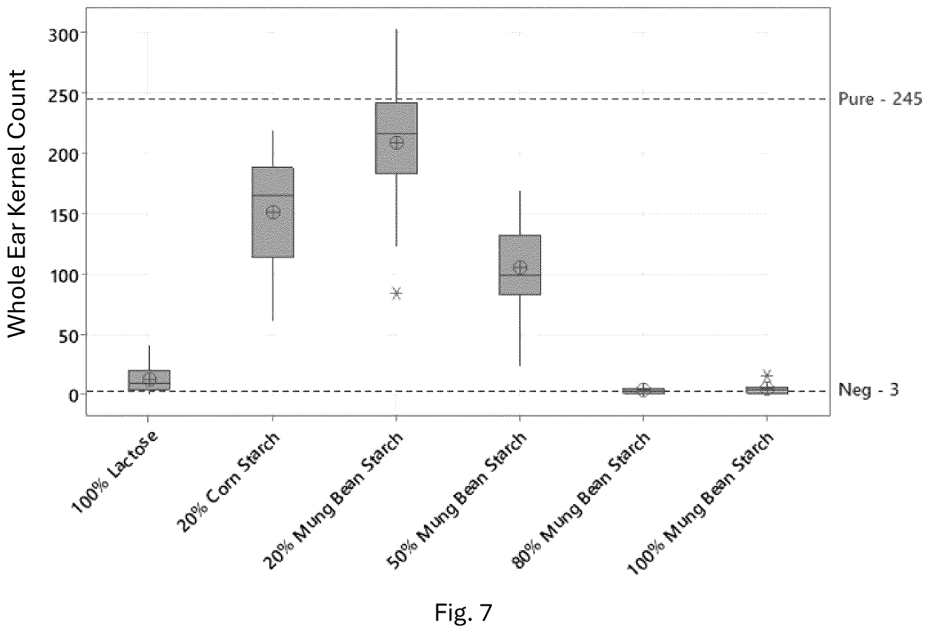

FIG. 7 shows seed set resulting from pollinations after three days of storage in diluent:pollen mixtures containing various proportions of mung bean starch compared to storage in lactose (0% starch) and lactose blended with 20% corn starch. Diluents containing 20% mung bean starch or corn starch were most favorable for maintaining pollen viability in storage. As observed in previous examples with diluent-starch mixtures, increasing the proportion of mung bean starch beyond 50% by weight was deleterious for maintaining pollen viability in storage. Surprisingly, the average kernels per ear using diluent containing 20% mung bean starch was nearly equal to the pure pollen control which received 4 times more pollen per ear. In many cases, kernels per ear exceeded that of the pure pollen controls. This novel result confirms the addition of starch derived from legume seed in lactose storage diluent effectively extends the viability of maize pollen in storage.

Example 8: Field evaluation of pollen-diluent mixtures including pea (Pisum sativum) starch.

This study was conducted on field-grown plants in Ankeny, Iowa during August 2024. The study objective was to evaluate the longevity of maize pollen stored in lactose diluents blended with pea starch. Fresh pollen collected mechanically from maize inbred WT944-6 was mixed with the various starch-blended diluents in a 4:1 (diluent:pollen) ratio by weight. Starch-based diluents included the following blends by weight: 80% lactose combined with 20% corn starch, 100% pea starch, 80% lactose combined with 20% pea starch, 50% lactose combined with 50% pea starch, and 20% lactose combined with 80% pea starch. Diluent:pollen mixtures were stored at 4° C. in 1 L storage vessels. After three days of storage, pollen-diluent mixtures were applied in metered (kernel limiting) doses onto exposed silks of male-sterile inbred WSS644 approximately three days after silks first emerged. Pollinations were performed by hand (scoop and tube technique using a 1/64^{th} teaspoon of pollen-diluent mixture) to 10 female plants per treatment/rep with three replications (n=30 per treatment, n=180 total)

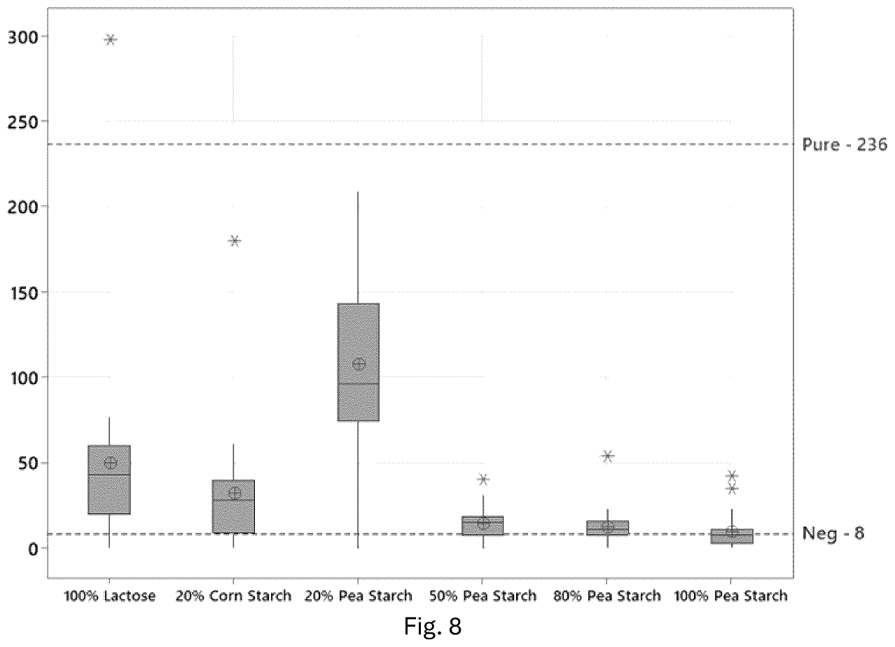

FIG. 8 shows seed set resulting from pollinations after three days of storage in diluent:pollen mixtures containing various proportions of pea starch compared to storage in lactose (0% starch) and lactose blended with 20% corn starch. Diluent containing 20% pea starch was most favorable for maintaining pollen viability in storage. Resulting kernels per ear were 2 to 3-fold greater than storage in lactose alone or diluent containing 20% corn starch, respectively. As observed in previous examples with diluent-starch mixtures, increasing the proportion of pea starch to 50% or greater by weight was deleterious for maintaining pollen viability in storage. This novel result confirms that the viability of maize pollen in storage can be extended dramatically by addition of starch derived from legume seed to the storage diluent.

1. A method of preserving Poaceae pollen comprising mixing collected Poaceae pollen with a diluent comprising between 10% and 50% starch granules by weight, wherein said preserved Poaceae pollen maintains its viability and can be used to successfully pollinate plants.

2. The method of claim 1 comprising storing said Poaceae pollen mixed with said diluent.

3. The method of claim 2 wherein said storage takes place at a temperature ranging from −196 degrees Celsius to −80 degrees Celsius.

4. The method of claim 2 wherein said storage takes place at a temperature ranging from −80 degrees Celsius to 0 degrees Celsius.

5. The method of claim 2 wherein said storage takes place at a temperature ranging from 0 degrees Celsius to 25 degrees Celsius.

6. The method of claim 1 wherein the diluent comprises 15% to 25% starch granules by weight.

7. The method of claim 1 wherein said Poaceae pollen is freshly collected.

8. The method of claim 1 wherein said Poaceae pollen has been previously collected and preserved.

9. The method of claim 1 wherein said Poaceae pollen is obtained from a single genetic source.

10. The method of claim 1 wherein said Poaceae pollen is obtained from multiple genetic sources and is combined prior to preservation.

11. The method of claim 1 wherein said Poaceae pollen and diluent mixture is applied to receptive plants.

12. The method of claim 1 wherein said starch has been extracted from a root vegetable.

13. The method of claim 12 wherein said root vegetable is selected from the group consisting of potato, sweet potato, cassava, taro, and yam.

14. The method of claim 1 wherein said starch has been extracted from a grain.

15. The method of claim 14 wherein said grain is selected from the group consisting of corn, barley, buckwheat, wheat, and rice.

16. The method of claim 1 wherein said starch is sago starch that has been extracted from a palm plant.

17. The method of claim 1 wherein said starch is arrowroot starch.

18. The method of claim 17 wherein said arrowroot starch has been extracted from a plant selected from the group consisting of Maranta arundinacea, Zamia integrifolia, Manihot esculenta, Tacca leontopetaloides, and Pueraria lobata.

19. The method of claim 1 wherein said starch has been extracted from a legume.

20. The method of claim 19 wherein said legume is selected from the group consisting of chickpea, cowpea, lentil, mung bean, pea, pigeon pea, fava bean, lima bean, and kidney bean.

21. The method of claim 1 wherein the remaining 50% to 90% of the diluent comprises a monosaccharide, disaccharide, or trisaccharide.

22. The method of claim 1 wherein the remaining 50% to 90% of the diluent is lactose.

23. The method of claim 1 wherein the preserved Poaceae pollen has enhanced performance when compared to preserved Poaceae pollen that has been stored in a similar diluent that is lacking the starch granules.

24. The method of claim 1 wherein the preserved Poaceae pollen has enhanced stability when compared to preserved Poaceae pollen that has been stored in a similar diluent that is lacking the starch granules.

25. A method of preserving Poaceae pollen comprising mixing collected Poaceae pollen with a diluent comprising between 10% and 50% starch granules by volume, wherein said preserved Poaceae pollen maintains its viability and can be used to successfully pollinate plants.

26. The method of claim 25 wherein the diluent comprises 15% to 25% starch granules by volume.

27. A composition comprising a population of Poaceae pollen grains mixed with a diluent, wherein the diluent comprises between 10% and 50% starch granules by weight.

28. The composition of claim 27 wherein said composition has been stored for a minimum of 12 hours prior to use in pollinating plants.

29. The composition of claim 27, wherein said composition comprises a diluent comprising between 15% and 25% starch granules by weight.

30. A composition comprising a population of Poaceae pollen grains mixed with a diluent comprising between 10% and 50% starch granules by volume.

31. The composition of claim 30 wherein said composition has been stored for a minimum of 12 hours prior to use in pollinating plants.

32. The composition of claim 30, wherein said composition comprises a diluent comprising between 15% and 25% starch granules by volume.
